(19) United States
(12) Patent Application Publication
(10) Pub. No.: US 2020/0385748 A1
(43) Pub. Date: Dec. 10, 2020

(54) COMPOSITIONS AND METHODS FOR DELIVERY OF A MACROMOLECULE OR MACROMOLECULAR COMPLEXES INTO A PLANT

(71) Applicant: Monsanto Technology LLC, St. Louis, MO (US)

(72) Inventors: Steven H. Schwartz, Davis, CA (US); Wei Zheng, Davis, CA (US)

(73) Assignee: Monsanto Technology LLC, St. Louis, MO (US)

(21) Appl. No.: 16/870,173

(22) Filed: May 8, 2020

Related U.S. Application Data
(60) Provisional application No. 62/845,095, filed on May 8, 2019.

(51) Int. Cl. C12N 15/82

(52) U.S. Cl. CPC C12N 15/8218

(57) ABSTRACT

Various compositions and methods for delivering a macromolecule or macromolecular complex into a plant cell are described. The processes for preparing these compositions are also described.

### CROSS-REFERENCE TO RELATED APPLICATIONS

This application claims benefit of U.S. provisional application Ser. No. 62/845,095, filed May 8, 2019, the entire disclosure of which is incorporated herein by reference.

### FIELD

The present invention relates to various compositions and methods for delivering a macromolecule or macromolecular complexes, such as a polynucleotide, a protein, or a ribonucleoprotein into a plant cell. The present invention further relates to processes for preparing these compositions.

### BACKGROUND

Initiation of RNA interference (RNAi) by topically applied polynucleotides has many applications including weed management and control of various plant diseases. To deliver polynucleotides and initiate RNAi in plants, several barriers need to be overcome. The first barrier to delivery is the cuticle, which covers parts of the plant above the ground surface. Stomatal flooding with spreading surfactants is one method of delivering agrochemicals into the plant. However, once inside the plant, a polynucleotide needs to pass through the cell wall and the plasma membrane. Thus, there remains a need for compositions and methods that facilitate the delivery of large macromolecules, such as polynucleotides or ribonucleoproteins, through plant cell walls and plasma membranes.

### BRIEF SUMMARY

Various embodiments are directed to compositions comprising a functionalized carbon quantum dot comprising a carbon quantum dot and a cationic polymer comprising one or more amine functional groups, wherein the cationic polymer has an average molecular weight of from about 1 kDa to about 15 kDa; and a polynucleotide for regulating or modulating the expression of a gene in a plant cell that is complexed with the functionalized carbon quantum dot, wherein the functionalized carbon quantum dot has a particle size that is no greater than about 15 nm. In some embodiments, compositions include dispersion compositions comprising the particulate composition as described herein, or a plurality thereof; a surfactant; and a solvent.

Several embodiments relate to compositions comprising a functionalized carbon quantum dot comprising a carbon quantum dot and a cationic polymer comprising one or more amine functional groups, wherein the cationic polymer has an average molecular weight of from about 1 kDa to about 15 kDa; and a ribonucleoprotein that is complexed with the functionalized carbon quantum dot, wherein the functionalized carbon quantum dot has a particle size that is no greater than about 15 nm. In some embodiments, compositions include dispersion compositions comprising the particulate composition as described herein, or a plurality thereof; a surfactant; and a solvent.

Various embodiments are directed to methods for delivering a macromolecule or macromolecular complex into a plant cell. Various embodiments are directed to a method of delivering a polynucleotide into a plant cell. Various embodiments are directed to a method of delivering a ribonucleoprotein into a plant cell. These methods generally comprise applying a dispersion composition as described herein, or dilution thereof onto a plant and/or a part thereof.

Several embodiments are also directed to various processes for preparing the compositions described herein. Some processes are directed to preparing a particulate composition as described herein. For example, certain processes comprises mixing a carbon quantum dot precursor compound and a cationic polymer comprising one or more amine functional groups and having an average molecular weight of from about 3 kDa to about 15 kDa to form a precursor mixture; carbonizing the carbon quantum dot precursor compound to form functionalized carbon quantum dots; and complexing one or more polynucleotides for regulating or modulating of a gene expression in a plant cell with the functionalized carbon quantum dots to form the particulate composition, wherein at least a portion of the functionalized carbon quantum dots have a particle size that is no greater than about 15 nm, no greater than about 12 nm, or no greater than about 10 nm. Other processes comprise carbonizing a carbon quantum dot precursor compound to form carbon quantum dots; mixing the carbon quantum dots with a cationic polymer comprising one or more amine functional groups and having an average molecular weight of from about 3 kDa to about 15 kDa to form functionalized carbon quantum dots; and complexing one or more polynucleotides for regulating or modulating the expression of a gene in a plant cell with the functionalized carbon quantum dots to form the particulate composition, wherein at least a portion of the functionalized carbon quantum dots have a particle size that is no greater than about 15 nm, no greater than about 12 nm, or no greater than about 10 nm. Other processes comprises mixing a carbon quantum dot precursor compound and a cationic polymer comprising one or more amine functional groups and having an average molecular weight of from about 3 kDa to about 15 kDa to form a precursor mixture; carbonizing the carbon quantum dot precursor compound to form functionalized carbon quantum dots; and complexing one or more ribonucleoprotein for modifying a target nucleotide sequence in a plant cell with the functionalized carbon quantum dots to form the particulate composition, wherein at least a portion of the functionalized carbon quantum dots have a particle size that is no greater than about 15 nm, no greater than about 12 nm, or no greater than about 10 nm. Other processes comprise carbonizing a carbon quantum dot precursor compound to form carbon quantum dots; mixing the carbon quantum dots with a cationic polymer comprising one or more amine functional groups and having an average molecular weight of from about 3 kDa to about 15 kDa to form functionalized carbon quantum dots; and complexing one or more ribonucleoproteins for modifying a nucleotide sequence in a plant cell with the functionalized carbon quantum dots to form the particulate composition, wherein at least a portion of the functionalized carbon quantum dots have a particle size that is no greater than about 15 nm, no greater than about 12 nm, or no greater than about 10 nm.

Other objects and features will be in part apparent and in part pointed out hereinafter.

### BRIEF DESCRIPTION OF THE DRAWINGS

FIG. 1 is an image of an agarose gel assay showing siRNA, alone or complexed with increasing concentrations of carbon dots, labeled with ethidium bromide.

FIG. 2 is an image of an agarose gel assay showing dsRNA, alone or complexed with carbon dots, after incubation with RNase for 5, 10 or 30 minutes. dsRNA that was not degraded by the RNase is visualized with ethidium bromide staining.

FIG. 3 shows the results of the quantitative RT-PCR analysis on GFP or MgChL mRNA messages after transfection with functionalized carbon dots in tomato leaves.

### DETAILED DESCRIPTION

The present invention relates to various compositions and methods for delivering a macromolecule or macromolecular complex from the exterior surface of a plant or plant part into the interior of a plant cell. In some embodiments, present invention relates to various compositions and methods for delivering a polynucleotide from the exterior surface of a plant or plant part into the interior of a plant cell. In some embodiments, present invention relates to various compositions and methods for delivering a protein from the exterior surface of a plant or plant part into the interior of a plant cell. In some embodiments, present invention relates to various compositions and methods for delivering a ribonucleoprotein from the exterior surface of a plant or plant part into the interior of a plant cell. In some embodiments, compositions of the present invention generally comprise a functionalized carbon quantum dot and a polynucleotide. In some embodiments, compositions of the present invention generally comprise a functionalized carbon quantum dot and a protein. In some embodiments, compositions of the present invention generally comprise a functionalized carbon quantum dot and a ribonucleoprotein. The present invention further relates to processes for preparing these compositions.

Various aspects of the present invention are directed to enhancing the delivery of polynucleotides into a plant cell, particularly for initiating RNAi or for gene editing. Common transfection agents in animal systems encapsulate nucleic acids in particles with sizes generally greater than 100 nm. However, the utility of these transfection agents in plants is complicated by the presence of a cell wall, which has a size exclusion limit that is much smaller than the size of the particles used for delivery. To address this problem, applicants have discovered that particulate compositions comprising certain carbon quantum dots can be particularly useful for delivering macromolecules, such as polynucleotides, through the plant cell wall and subsequent barriers.

Several embodiments of the present invention are directed to enhancing the stability of polynucleotides for delivery into a plant cell. Once applied to a plant, polynucleotides may be degraded by nucleases. It has been discovered that complexing polynucleotides with carbon quantum dots may provide for enhanced resistance to nucleases. This discovery could significantly improve efficacy in plants, particularly in those which contain a significant amount of nuclease activity in extracellular apoplast.

Several embodiments of the present invention are directed to enhancing the stability of polynucleotides for delivery to an insect. In some embodiments, compositions as described herein are applied to a plant upon which the insect feeds.

Other aspects of the present invention are directed to the initiation of RNAi or gene editing with topically applied polynucleotides at relatively lower concentrations. As noted, complexing polynucleotides with carbon quantum dots can enhance delivery through the cell wall and enhance resistance to nucleases. As a result, a relatively lower concentration of the polynucleotide may be required to initiate RNAi or induce gene editing. Compositions that require a relatively lower concentration of RNAi are especially beneficial for reducing costs associated with large-scale application of polynucleotides for agricultural uses.

### I. Particulate Compositions

Various compositions of the present invention include particulate compositions comprising a carbon quantum dot and a macromolecule or macromolecular complex. In some embodiments, the particulate compositions comprise (1) a functionalized carbon quantum dot comprising a carbon quantum dot and a cationic polymer and (2) a macromolecule or macromolecular complex. In these compositions, the macromolecule or macromolecular complex is typically complexed with the functionalized carbon quantum dot. In some embodiments, compositions as described herein may further comprise one or more agents for conditioning of a plant to permeation by a carbon quantum dot and a macromolecule or macromolecular complex. Such permeation conditioning agents include, e. g., surfactants, organic solvents, aqueous solutions or aqueous mixtures of organic solvents, oxidizing agents, acids, bases, oils, enzymes, or combinations thereof.

Various compositions of the present invention include particulate compositions comprising a carbon quantum dot and a polynucleotide. In some embodiments, the particulate compositions comprise (1) a functionalized carbon quantum dot comprising a carbon quantum dot and a cationic polymer and (2) a polynucleotide, particularly a polynucleotide. In some embodiments, the polynucleotide regulates or modulates expression of a gene in a plant cell. In some embodiments, the polynucleotide is a guide RNA. In these compositions, the polynucleotide is typically complexed with the functionalized carbon quantum dot.

Various compositions of the present invention include particulate compositions comprising a carbon quantum dot and a ribonucleoprotein. In some embodiments, the particulate compositions comprise (1) a functionalized carbon quantum dot comprising a carbon quantum dot and a cationic polymer and (2) a ribonucleoprotein. In some embodiments, the ribonucleoprotein comprises a CRISPR associated protein and a guide RNA. In these compositions, the ribonucleoprotein is typically complexed with the functionalized carbon quantum dot.

Functionalized Carbon Quantum Dot

As noted, the functionalized carbon quantum dot comprises a carbon quantum dot. Generally, carbon quantum dots can be synthesized by various techniques. In some techniques, carbon quantum dots are synthesized by a “top down” approach. In these techniques, carbon quantum dots are formed during the production of larger structured carbon precursors such as graphene. In other techniques, carbon quantum dots are synthesized by a “bottom-up” approach from simple carbon-based precursors. In these techniques, a carbon quantum dot precursor compound is heated at elevated temperature such as from about 75° C. to about 300° C., from about 75° C. to about 200° C., from about 100° C. to about 300° C. or from about 100° C. to about 200° C. to carbonize the precursor, thereby forming the carbon quantum dot. Heating can be conducted by various means. For example, heating can be conducted via microwave methods, heating in an autoclave, or refluxing in a solvent. After synthesis, carbon quantum dots can be purified or fractionated by ultrafiltration, dialysis, size exclusion chromatography, and combinations thereof to remove unreacted precursors and by-products.

As noted, the carbon quantum dot can comprise a carbonization product of at least one carbon quantum dot precursor compound. Carbon quantum dot precursor compounds include, for example, various polyols, organic acids, saccharides, azoles, azines, and combinations of these compounds.

Polyols include various diols, triols, tetrols, and so on, as well as alkoxylated polyols, and any combinations of these. Specific examples of polyols include glycerol, ethylene glycol, and polyethylene glycols. Organic acids include, for example, various mono-, di-, and tri-carboxyylic acids, and combinations thereof. Specific examples of organic acids include citric acid, C₂-C₂₀ mono- and di-carboxylic acids such as C₂-C₂₀ aldonic acids, C₂-C₂₀ aldaric acids, and related linear C₂-C₂₀ mono- and di-carboxylic acids such as succinic acid and adipic acid. Saccharides include various monosaccharides, disaccharides, oligosaccharides, etc. Particular examples of saccharides include glucose, fructose, and lactose. Saccharride derivatives include, for example, various amine-substituted saccharides such as glucosamine. Azoles and azines include various 5- and 6-membered nitrogen-containing aromatic ring compounds such as imidazole, pyridine, and pyrazine.

In some embodiments, the carbon quantum dot comprises a carbonization product of at least one carbon quantum dot precursor compound selected from the group consisting of a polyol, a saccharide, a saccharide derivative, and combinations thereof. In certain embodiments, the carbon quantum dot comprises a carbonization product of at least one carbon quantum dot precursor compound selected from the group consisting of glucose, fructose, lactose, glucosamine, glycerol, ethylene glycol, polyethylene glycol and combinations thereof. In further embodiments, the carbon quantum dot comprises a carbonization product of at least one carbon quantum dot precursor compound comprising a polyethylene glycol have having an average molecular weight of from about 100 Da to about 500 Da, from about 100 Da to 400 Da, or from about 150 Da to about 250 Da.

In certain embodiments, the carbon quantum dot and/or the carbon quantum dot precursor compound is essentially free or free of sulfur. For example, in some embodiments the carbon quantum dot precursor compound does not include a sulfur atom or sulfur-containing moiety.

As noted, the functionalized carbon quantum dot also comprises a cationic polymer. Functionalization of the carbon quantum dots with cationic polymers can increase the colloidal stability of the carbon quantum dot and provides for binding or complexing of the macromolecule or macromolecular complex. In some embodiments, the macromolecule is selected from a protein or a polynucleotide. In some embodiments, the macromolecular complex is a ribonucleoprotein comprising a CRISPR associate protein and a guide RNA. Typically, the cationic polymer comprises one or more amine functional groups. In various embodiments, the cationic polymer comprising one or more amine functional groups includes, for example, polyethyleneimines (PEIs), polydiallyldimethylammonium (PDDA) polymer, and polybrene (1,5-dimethyl-1,5-diazaundecamethylene polymethobromide).

Polyethyleneimines can be linear or branched. It has been discovered that, in some instances, branched polyethyleneimines can provide for improved efficacy (e.g., gene regulations or modulation) as compared to linear polyethyleneimines. Accordingly, in some embodiments, the cationic polymer comprises a branch polyethyleneimine. In certain embodiments, the cationic polymer consists essentially of one or more polyethyleneimines (e.g., at least about 95 wt. %, at least about 95 wt. %, or at least about 99 wt. % of the cationic polymer consists of one or more polyethyleneimines). In select embodiments, the cationic polymer consists of one or more polyethyleneimines.

The molecular weight of the cationic polymer has been found to be one factor that affects the activity of the functionalized carbon quantum dot. In various, embodiments, the cationic polymer has an average molecular weight of from about 1 kDa to about 15 kDa, from about 3 kDa to about 15 kDa, from about 4 kDa to about 12 kDa, or from about 5 kDa to about 10 kDa. In some embodiments, the cationic polymer has an average molecular weight of about 1 kDa, about 2 kDa, about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, or about 15 kDa.

The cationic polymer can be comprised of a mixture of two or more polymers. For example, in some embodiments, the cationic polymer comprises a mix of two or more polymers having different average molecular weights.

Typically, the functionalized carbon quantum dot has a particle size that is less than the size exclusion limit of a plant cell wall. Accordingly, in various embodiments, the functionalized carbon quantum dot has a particle size (i.e., particle diameter) that is no greater than about 15 nm, no greater than about 12 nm, or no greater than about 10 nm. For example, the functionalized carbon quantum dot can have a particle size that is from about 0.5 nm to about 15 nm, from about 0.5 nm to about 12 nm, from about 0.5 nm to about 10 nm, from about 0.5 nm to about 8 nm, from about 1 nm to about 15 nm, from about 1 nm to about 12 nm, from about 1 nm to about 10 nm, from about 1 nm to about 8 nm, from about 5 nm to about 15 nm, from about 5 nm to about 12 nm, from about 5 nm to about 10 nm, or from about 5 nm to about 8 nm. Particle size can be measured by dynamic light scattering (DLS), transmission electron microscopy (TEM), atomic force microscopy (AFM), or size exclusion chromatography (SEC). Preferably, particle size may be measured by dynamic light scattering (DLS) or size exclusion chromatography (SEC).

The particle size of the particulate composition comprising the functionalized carbon quantum dot and the macromolecule or macromolecular complex can be approximately the same as the particle size of the functionalized carbon quantum dot. In other embodiments, the particle size of the particulate composition comprising the functionalized carbon quantum dot and the macromolecule or macromolecular complex can be approximately the 3 to 6 nm greater than the particle size of the functionalized carbon quantum dot. In some embodiments, the macromolecule is selected from a protein or a polynucleotide. In some embodiments, the macromolecular complex is a ribonucleoprotein comprising a CRISPR associate protein and a guide RNA. Thus, the particulate composition can have a particle size that is no greater than about 21 nm, no greater than about 18 nm, no greater than about 15 nm, no greater than about 12 nm, or no greater than about 10 nm. For example, the particulate composition can have a particle size that is from about 0.5 nm to about 21 nm, from about 0.5 nm to about 18 nm, from about 0.5 nm to about 15 nm, from about 0.5 nm to about 12 nm, from about 0.5 nm to about 10 nm, from about 0.5 nm to about 8 nm, from about 1 nm to about 21 nm, from about 1 nm to about 18 nm, from about 1 nm to about 15 nm, from about 1 nm to about 12 nm, from about 1 nm to about 10 nm, from about 1 nm to about 8 nm, from about 5 nm to about 21 nm, from about 5 nm to about 18 nm, from about 5 nm to about 15 nm, from about 5 nm to about 12 nm, from about 5 nm to about 10 nm, or from about 5 nm to about 8 nm.

Various processes can be used to prepare the functionalized carbon quantum dots. Some processes comprise mixing a carbon quantum dot precursor compound as described herein and a cationic polymer (e.g., a cationic polymer comprising one or more amine functional groups and having an average molecular weight of from about 3 kDa to about 15 kDa) to form a precursor mixture and carbonizing the carbon quantum dot precursor compound to form functionalized carbon quantum dots. In other processes, the carbon quantum dot is formed first and then functionalized. These processes comprise carbonizing a carbon quantum dot precursor compound as described herein to form carbon quantum dots and mixing the carbon quantum dots with a cationic polymer (e.g., a cationic polymer comprising one or more amine functional groups and having an average molecular weight of from about 3 kDa to about 15 kDa) to form the functionalized carbon quantum dots.

Polynucleotides

In addition to a functionalized carbon quantum dot, the particulate compositions of the present invention also comprise a polynucleotide. In some embodiments, polynucleotides described herein may be useful for regulating or modulating the expression of a gene in a plant cell or may be used to express a non-native protein in the cell (e.g., a nuclease to induce genetic alterations in the plant cell and/or a non-native protein that can confer a beneficial property to the plant). In some embodiments, the polynucleotides described herein may be useful for for guiding a CRISPR associate protein to a target nucleotide sequence. As noted, the polynucleotide is complexed with the functionalized carbon quantum dot.

The term “polynucleotide” refers to a nucleic acid molecule containing multiple nucleotides and generally refers both to “oligonucleotides” (a polynucleotide molecule of 18-25 nucleotides in length) and polynucleotides of 26 or more nucleotides. Polynucleotides also include molecules containing multiple nucleotides including non-canonical nucleotides or chemically modified nucleotides as commonly practiced in the art; see, e.g., chemical modifications disclosed in the technical manual “RNA Interference (RNAi) and DsiRNAs”, 2011 (Integrated DNA Technologies Coralville, Iowa).

Polynucleotides to Modify Gene Expression

When used to regulate or modulate expression of a gene in a plant cell, the polynucleotides can be DNA or RNA or both, can be either single- or double-stranded, and can include at least one segment of 10 or more or 18 or more contiguous nucleotides (or, in the case of double-stranded polynucleotides, at least 10 or at least 18 contiguous base-pairs) that are essentially identical, essentially complementary or having a high degree of similarity or complementarity to a fragment of equivalent size of the DNA of a target gene or the target gene's RNA transcript. In various embodiments, the polynucleotide has a length of 16-25 nucleotides (e.g., 16-mers, 17-mers, 18-mers, 19-mers, 20-mers, 21-mers, 22-mers, 23-mers, 24-mers, or 25-mers), or medium-length polynucleotides having a length of 26 or more nucleotides (e.g., polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), or long polynucleotides having a length at least about 300 nucleotides (e.g., polynucleotides of from about 300 to about 400 nucleotides, from about 400 to about 500 nucleotides, from about 500 to about 600 nucleotides, from about 600 to about 700 nucleotides, from about 700 to about 800 nucleotides, from about 800 to about 900 nucleotides, from about 900 to about 1000 nucleotides, from about 300 to about 500 nucleotides, from about 300 to about 600 nucleotides, from about 300 to about 700 nucleotides, from about 300 to about 800 nucleotides, from about 300 to about 900 nucleotides, or about 1000 nucleotides in length, or even greater than about 1000 nucleotides in length, for example, up to 2000 nucleotides, 3000 nucleotides, 4000 nucleotides, 5000 nucleotides in length, or up to the entire length of a target gene including coding or non-coding or both coding and non-coding portions of the target gene). Where a polynucleotide is double-stranded, its length can be similarly described in terms of base pairs.

The polynucleotides described herein can be single-stranded (ss) or double-stranded (ds). “Double-stranded” refers to the base-pairing that occurs between sufficiently complementary, anti-parallel nucleic acid strands to form a double-stranded nucleic acid structure, generally under physiologically relevant conditions. Embodiments include those wherein the polynucleotide is selected from the group consisting of sense single-stranded DNA (ssDNA), sense single-stranded RNA (ssRNA), double-stranded RNA (dsRNA), double-stranded DNA (dsDNA), a double-stranded DNA/RNA hybrid, anti-sense ssDNA, or anti-sense ssRNA; a mixture of polynucleotides of any of these types can be used. In various embodiments, the polynucleotide is selected from the group consisting of single-stranded DNA (ssDNA), single-stranded RNA (ssRNA), double-stranded DNA (dsDNA), double-stranded RNA (dsRNA), and RNA/DNA hybrid.

In certain embodiments, the polynucleotide is dsRNA. In some embodiments, the polynucleotide is dsRNA of at least about 10 contiguous base pairs in length. In some embodiments, the polynucleotide is dsRNA with a length of from about 10 to about 500 base pairs, from about 16 to about 400 base pairs, from about 18 to about 300 base pairs, from about 18 to about 200 base pairs, or from about 18 to about 50 base pairs.

As used herein, “dsRNA” refers to a molecule comprising two antiparallel ribonucleotide strands bound together by hydrogen bonds, each strand of which comprises ribonucleotides linked by phosphodiester bonds running in the 5′-3′ direction. Two antiparallel strands of a dsRNA can be perfectly complementary to each other or comprise one or more mismatches up to a degree where any one additional mismatch causes the disassociation of the two antiparallel strands. A dsRNA molecule can have perfect complementarity over the entire dsRNA molecule, or comprises only a portion of the entire molecule in a dsRNA configuration. An RNA molecule containing inverted repeats can also form a dsRNA structure, e.g., a hairpin like structure (often also called a stem-loop structure).

In some embodiments, the polynucleotide is a microRNA (miRNA), miRNA decoy (e.g., as disclosed in U.S. Patent Application Publication 2009/0070898 which is incorporated herein by reference), a miRNA precursor, or a transacting RNA (ta-siRNA). In some embodiments, the polynucleotide is double-stranded RNA of a length greater than that which is typical of naturally occurring regulatory small RNAs (such as endogenously produced siRNAs and mature miRNAs).

In various embodiments, the polynucleotide can include components other than standard ribonucleotides, e.g., an embodiment is an RNA that comprises terminal deoxyribonucleotides.

Various embodiments relate to a polynucleotide comprising at least one segment of 18 or more contiguous nucleotides with a sequence of about 80%, about 81%, about 82%, about 83%, about 84%, about 85%, about 86%, about 87%, about 88%, about 89%, about 90%, about 91%, about 92%, about 93%, about 94%, or about 95% to about 100% identity with a fragment of equivalent length of a DNA of a target gene. In some embodiments, the contiguous nucleotides number at least 16, e.g., from 16 to 24, or from 16 to 25, or from 16 to 26, or from 16 to 27, or from 16 to 28. In certain embodiments, the contiguous nucleotides number at least 18, e.g., from 18 to 24, or from 18 to 28, or from 20 to 30, or from 20 to 50, or from 20 to 100, or from 50 to 100, or from 50 to 500, or from 100 to 250, or from 100 to 500, or from 200 to 1000, or from 500 to 2000, or even greater. In further embodiments, the contiguous nucleotides number more than 16, e.g., 17, 18, 19, 20, 21, 22, 23, 24, 25, 26, 27, 28, 29, 30, or greater than 30, e.g., about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 350, about 400, about 450, about 500, about 600, about 700, about 800, about 900, about 1000, or greater than 1000 contiguous nucleotides. In still further embodiments, the polynucleotide comprises at least one segment of at least 21 contiguous nucleotides with a sequence of 100% identity with a fragment of equivalent length of a DNA of a target gene. In some embodiments, the polynucleotide is a double-stranded nucleic acid (e.g., dsRNA) with one strand comprising at least one segment of at least 21 contiguous nucleotides with 100% identity with a fragment of equivalent length of a DNA of a target gene; expressed as base-pairs, such a double-stranded nucleic acid comprises at least one segment of at least 21 contiguous, perfectly matched base-pairs which correspond to a fragment of equivalent length of a DNA of a target gene, or the DNA complement thereof. In various embodiments, each segment contained in the polynucleotide is of a length greater than that which is typical of naturally occurring regulatory small RNAs, for example, each segment is at least about 30 contiguous nucleotides (or base-pairs) in length.

As used herein, the terms “homology” and “identity” when used in relation to nucleic acids, describe the degree of similarity between two or more nucleotide sequences. The percentage of “sequence identity” between two sequences is determined by comparing two optimally aligned sequences over a comparison window, such that the portion of the sequence in the comparison window may comprise additions or deletions (gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. An alignment of two or more sequences may be performed using any suitable computer program. For example, a widely used and accepted computer program for performing sequence alignments is CLUSTALW v1.6 (Thompson, et al. Nucl. Acids Res., 22: 4673-4680, 1994).

As used herein, the term “essentially identical” or “essentially complementary” means that the polynucleotide (or at least one strand of a double-stranded polynucleotide or portion thereof, or a portion of a single strand polynucleotide) hybridizes under physiological conditions to the target gene, an RNA transcribed there from, or a fragment thereof, to effect regulation or suppression of the target gene. For example, in some embodiments, a polynucleotide has 100 percent sequence identity or at least about 80, 81, 82, 83, 84, 85, 86, 87, 88, 89, 90, 91, 92, 93, 94, 95, 96, 97, 98, or 99 percent sequence identity when compared to a sequence of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more contiguous nucleotides in the target gene or RNA transcribed from the target gene. In some embodiments, a polynucleotide has 100 percent sequence complementarity or at least about 80, 81, 82, 83, 84, 85, 86, 87, 88, 89, 90, 91, 92, 93, 94, 95, 96, 97, 98, or 99 percent sequence complementarity when compared to a sequence of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more contiguous nucleotides in the target gene or RNA transcribed from the target gene. In some embodiments, a polynucleotide has 100 percent sequence identity with or complementarity to one allele or one family member of a given target gene (coding or non-coding sequence of a gene). In some embodiments, a polynucleotide has at least about 80, 81, 82, 83, 84, 85, 86, 87, 88, 89, 90, 91, 92, 93, 94, 95, 96, 97, 98, or 99 percent sequence identity with or complementarity to multiple alleles or family members of a given target gene. In some embodiments, a polynucleotide has 100 percent sequence identity with or complementarity to multiple alleles or family members of a given target gene.

In various embodiments, the polynucleotide described herein comprises naturally occurring nucleotides, such as those which occur in DNA and RNA. In certain embodiments, the polynucleotide is a combination of ribonucleotides and deoxyribonucleotides, for example, synthetic polynucleotides consisting mainly of ribonucleotides but with one or more terminal deoxyribonucleotides or one or more terminal dideoxyribonucleotides or synthetic polynucleotides consisting mainly of deoxyribonucleotides but with one or more terminal dideoxyribonucleotides. In certain embodiments, the polynucleotide comprises non-canonical nucleotides such as inosine, thiouridine, or pseudouridine. In certain embodiments, the polynucleotide comprises chemically modified nucleotides. Examples of chemically modified oligonucleotides or polynucleotides are well known in the art; see, for example, U.S. Patent Application Publications 2011/0171287, 2011/0171176, 2011/0152353, 2011/0152346, and 2011/0160082, which are herein incorporated by reference. Illustrative examples include, but are not limited to, the naturally occurring phosphodiester backbone of an oligonucleotide or polynucleotide which can be partially or completely modified with phosphorothioate, phosphorodithioate, or methylphosphonate internucleotide linkage modifications, modified nucleoside bases or modified sugars can be used in oligonucleotide or polynucleotide synthesis, and oligonucleotides or polynucleotides can be labeled with a fluorescent moiety (e.g., fluorescein or rhodamine) or other label (e.g., biotin).

In various embodiments, the polynucleotide is a non-transcribable polynucleotide. The term “non-transcribable polynucleotide” refers to a polynucleotide that does not comprise a complete polymerase II transcription unit. In other embodiments, the polynucleotide is a transcribable polynucleotide. For example, in some embodiments the polynucleotide may be transcribed to express a protein not naturally found in the organism. In other embodiments, the polynucleotide may be transcribed to express a genome editing protein. In some embodiments, the polynucleotide is a plasmid or a viral vector.

In various embodiments, the polynucleotide is a polynucleotide designed to modulate or regulate the expression of a target gene. In some embodiments the polynucleotide is a bioactive polynucleotide molecule comprises a nucleotide sequence that is substantially homologous or complementary to a polynucleotide sequence of a target gene or an RNA expressed from the target gene or a fragment thereof and functions to suppress the expression of the target gene or produce a knock-down phenotype. In some embodiments, polynucleotides are capable of inhibiting or “silencing” the expression of a target gene and are generally described in relation to their “target sequence.” Such polynucleotides may be single-stranded DNA (ssDNA), single-stranded RNA (ssRNA), double-stranded RNA (dsRNA), double-stranded DNA (dsDNA), or double-stranded DNA/RNA hybrids; and may comprise naturally-occurring nucleotides, modified nucleotides, nucleotide analogues or any combination thereof. In some embodiments, a polynucleotide designed to modulate or regulate the expression of a target gene may be incorporated within a larger polynucleotide. In certain embodiments, a polynucleotide may be processed into a small interfering RNA (siRNA).

As used herein, the terms “target gene” or “target sequence” or “target nucleic acid sequence” refer to a nucleotide sequence that occurs in a gene or gene product against which a polynucleotide is directed. In this context, the term “gene” means a locatable region of genomic sequence, corresponding to a unit of inheritance, which includes regulatory regions, such as promoters, enhancers, 5′ untranslated regions, intron regions, 3′ untranslated regions, transcribed regions, and other functional sequence regions that may exist as native genes or transgenes in a plant genome or the genome of a pathogen. As used herein, the term “pathogen” refers to virus, viroid, bacteria, fungus, oomycetes, protozoa, phytoplasma, and parasitic plants. Depending upon the circumstances, the terms target sequence or target gene or target nucleic acid sequence can refer to the full-length nucleotide sequence of the gene or gene product targeted for suppression or the nucleotide sequence of a portion of the gene or gene product targeted for suppression. Depending upon the circumstances, the terms target sequence or target gene or target nucleic acid sequence can refer to a nucleotide sequence targeted for modification by a genome editing protein.

The target gene can be an endogenous gene, a viral gene or a transgene. The target gene can be an endogenous plant gene, a transgene expressed in a plant cell, an endogenous gene of a plant pathogen, an essential gene of an insect, or a transgene expressed in a plant pathogen. The term “pathogen” refers to virus, viroid, bacteria, fungus, oomycetes, protozoa, phytoplasma, and parasitic plants. In some embodiments, the target gene 1) is an essential gene for maintaining the growth and life of the plant; 2) encodes a protein that provides herbicide resistance to the plant; or 3) transcribes to an RNA regulatory agent. In some embodiments, the target gene is exogenous to the plant in which the polynucleotide is to be introduced, but endogenous to a plant pathogen.

The target gene can be translatable (coding) sequence, or can be a non-coding sequence (such as non-coding regulatory sequence), or both. Examples of a target gene include non-translatable (non-coding) sequence, such as, but not limited to, 5 ‘ untranslated regions, promoters, enhancers, or other non-coding transcriptional regions, 3’ untranslated regions, terminators, and introns. Target genes include genes encoding microRNAs, small interfering RNAs, and other small RNAs associated with a silencing complex (RISC) or an Argonaute protein; RNA components of ribosomes or ribozymes; small nucleolar RNAs; and other non-coding RNAs. Target genes can also include genes encoding transcription factors and genes encoding enzymes involved in the biosynthesis or catabolism of molecules of interest (such as, but not limited to, amino acids, fatty acids and other lipids, sugars and other carbohydrates, biological polymers, and secondary metabolites including alkaloids, terpenoids, polyketides, non-ribosomal peptides, and secondary metabolites of mixed biosynthetic origin).

The target gene can include a single gene or part of a single gene that is targeted for suppression, or can include, for example, multiple consecutive segments of a target gene, multiple non-consecutive segments of a target gene, multiple alleles of a target gene, or multiple target genes from one or more species.

In some embodiments, the polynucleotide is useful for transiently silencing one or more genes in a cell of a growing plant or whole plant to affect a desired phenotype in response to culture conditions, environmental or abiotic or biotic stress, herbicide exposure, or change in market demand during the growing season or in the post-harvest environment. For example, the polynucleotide is useful for transiently suppressing a biosynthetic or catabolic gene in order to produce a plant or plant product with a desired phenotype, such as a desired nutritional composition of a crop plant product, e.g., suppressing a FAD2 gene to affect a desired fatty acid profile in soybean or canola or other oilseed or suppressing a lignin biosynthetic genes such as COMT and CCOMT to provide more easily digestible forage plants.

Target genes can include genes encoding herbicide-tolerance proteins, non-coding sequences including regulatory RNAs, and essential genes, which are genes necessary for sustaining cellular life or to support reproduction of an organism.

In some embodiments, the polynucleotide is useful for silencing one or more essential genes in a plant. Embodiments of essential genes include genes involved in DNA or RNA replication, gene transcription, RNA-mediated gene regulation, protein synthesis, energy production, and cell division. One example of a compendium of essential genes in plants is described in Zhang et al. (2004) Nucleic Acids Res., 32:D271-D272, version DEG 5.4 lists 777 essential genes for Arabidopsis thaliana. Examples of essential genes include translation initiation factor (TIF) and ribulose-1,5-bisphosphate carboxylase oxygenase (RuBisCO). Target genes can include genes encoding transcription factors and genes encoding enzymes involved in the biosynthesis or catabolism of molecules in plants such as, but not limited to, amino acids, fatty acids and other lipids, sugars and other carbohydrates, biological polymers, and secondary metabolites including alkaloids, terpenoids, polyketides, non-ribosomal peptides, and secondary metabolites of mixed biosynthetic origin. Specific examples of suitable target genes also include genes involved in amino acid or fatty acid synthesis, storage, or catabolism, genes involved in multi-step biosynthesis pathways, where it may be of interest to regulate the level of one or more intermediate; and genes encoding cell-cycle control proteins. Target genes can include genes encoding undesirable proteins (e.g., allergens or toxins) or the enzymes for the biosynthesis of undesirable compounds (e.g., undesirable flavor or odor components). In some embodiments, the polynucleotide is useful for silencing one or more essential genes in an insect. Embodiments of essential genes include genes involved in DNA or RNA replication, gene transcription, RNA-mediated gene regulation, protein synthesis, energy production, and cell division. In some embodiments, the essential gene is selected from the group consisting of Act5C, arginine kinase, COPI (coatomer subunit) alpha, COPI (coatomer subunit) beta, COPI (coatomer subunit) betaPrime, COPI (coatomer subunit) delta, COPI (coatomer subunit) epsilon, COPI (coatomer subunit) gamma, COPI (coatomer subunit) zeta, RpL07, RpL19, RpL3, RpL40, RpS21, RpS4, Rpn2, Rpn3, Rpt6, Rpn8, Rpn9, Rpn6-PB-like protein, Sarl, sec6, sec23, sec23A, shrb (snf7), Tubulin gamma chain, ProsAlpha2, ProsBeta5, Proteasome alpha 2, Proteasome beta 5, VATPase E, VATPase A, VATPase B, VATPase D, Vps2, Vps4, Vps16A, Vps20, Vps24, Vps27, Vps28, Vha26 (V-ATPase A), Vha68-2 (V-ATPase D/E), 40S ribosomal protein S14, and 60S ribosomal protein L13.

Target genes might also include essential genes of a plant pathogen. Essential genes include genes that, when silenced or suppressed, result in the death of the pathogen or in the pathogen's inability to successfully reproduce. In some embodiments, the target gene is a sequence from a pathogenic virus. Examples of fungal plant pathogens include, e.g., the fungi that cause powdery mildew, rust, leaf spot and blight, damping-off, root rot, crown rot, cotton boll rot, stem canker, twig canker, vascular wilt, smut, or mold, including, but not limited to, Fusarium spp., Phakospora spp., Rhizoctonia spp., Aspergillus spp., Gibber ella spp., Pyricularia spp., and Alternaria spp., and the numerous fungal species provided in Tables 4 and 5 of U.S. Pat. No. 6,194,636, which is specifically incorporated in its entirety by reference herein. Examples of plant pathogens include pathogens previously classified as fungi but more recently classified as oomycetes. Specific examples of oomycete plant pathogens of particular interest include members of the genus Pythium (e.g., Pythium aphanidermatum) and Phytophthora (e.g., Phytophthora infestans, Phytophthora sojae) and organisms that cause downy mildew (e.g., Peronospora farinosa).

Effective polynucleotides of any size can be used, alone or in combination, in the various methods and compositions described herein. In some embodiments, polynucleotides comprising the same sequence is used to make a composition (e.g., a composition for topical application, or a recombinant DNA construct useful for making a transgenic plant). In other embodiments, a mixture or pool of different polynucleotides is used; in such cases the polynucleotides can be for a single target gene or for multiple target genes.

It will be appreciated that a polynucleotide, for example dsRNA, of the present disclosure need not be limited to those molecules containing only natural nucleotides, but further encompasses chemically-modified nucleotides and non-nucleotides. Polynucleotides of the present disclosure may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia of Polymer Science And Engineering, pages 858-859, Kroschwitz, J. I., ed. John Wiley & Sons, 1990, those disclosed by Englisch et al., Angewandte Chemie, International Edition, 1991, 613, and those disclosed by Sanghvi, Y. S., Chapter 15, Antisense Research and Applications, pages 289-2, Crooke, S. T. and Lebleu, B., ed., CRC Press, 1993. Such bases are particularly useful for increasing the binding affinity of the oligomeric compounds of the disclosure. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi Y S et al. (1993) Antisense Research and Applications, CRC Press, Boca Raton 276-278) and are presently preferred base substitutions, even more particularly when combined with 2′-0-methoxyethyl sugar modifications.

Delivery of Gene Editing Components

In several embodiments, the compositions and methods described herein may be utilized to deliver gene editing components to plant cells. In some embodiments, macromolecules or macromolecular complexes as described herein can be used to induce changes in the genome of the plant or plant cell (e.g., by inducing direct genetic modifications). Macromolecules and macromolecular complexes suitable for these gene editing applications are described in more detail herein. It will be appreciated by one of skill in the art that when any method or application described herein requires the delivery of a macromolecule or a macromolecular complex into a cell, that a composition comprising a functionalized carbon dot complexed to the macromolecule or a macromolecular complex may be formulated for delivery into a cell according to the methods described in Section II, below.

Genome Editing

Targeted modification of plant genomes through the use of genome editing methods can be used to create improved plant lines through modification of plant genomic DNA. In addition, genome editing methods can enable targeted insertion of one or more nucleic acids of interest into a plant genome. Example methods for introducing donor polynucleotides into a plant genome or modifying genomic DNA of a plant include the use of sequence specific nucleases, such as zinc-finger nucleases, engineered or native meganucleases, TALE-endonucleases, or an RNA-guided endonucleases (for example, a Clustered Regularly Interspersed Short Palindromic Repeat (CRISPR)/Cas9 system, a CRISPR/Cpf1 system, a CRISPR/CasX system, a CRISPR/CasY system, a CRISPR/Cascade system). Several embodiments relate to methods of genome editing is using single-stranded oligonucleotides to introduce precise base pair modifications in a plant genome, as described by Sauer et al (Plant Physiol. 2016 April; 170(4): 1917-1928). Methods of genome editing to modify, delete, or insert nucleic acid sequences into genomic DNA are known in the art.

Several embodiments relate to compositions and methods for delivery of a CRISPR/Cas9 system used to modify or replace an existing coding sequence within a plant genome. Several embodiments relate to compositions and methods for delivery of a CRISPR/Cpf1 system used to modify or replace an existing coding sequence within a plant genome. In further embodiments, compositions and methods for delivery of transcription activator-like effectors (TALEs) are used for modification or replacement of an existing coding sequence within a plant genome. In some embodiments, an existing polypeptide coding sequence within a plant genome is modified by non-templated genome editing with a sequence specific nuclease. In some embodiments, an existing polypeptide coding sequence within a plant genome is modified by templated genome editing with a sequence specific nuclease.

In an aspect, a “modification” comprises the insertion of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In another aspect, a “modification” comprises the deletion of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In a further aspect, a “modification” comprises the inversion of at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In still another aspect, a “modification” comprises the substitution of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In some embodiments, a “modification” comprises the substitution of an “A” for a “C”, “G” or “T” in a nucleic acid sequence. In some embodiments, a “modification” comprises the substitution of a “C” for an “A”, “G” or “T” in a nucleic acid sequence. In some embodiments, a “modification” comprises the substitution of a “G” for an “A”, “C” or “T” in a nucleic acid sequence. In some embodiments, a “modification” comprises the substitution of a “T” for an “A”, “C” or “G” in a nucleic acid sequence. In some embodiments, a “modification” comprises the substitution of a “C” for a “U” in a nucleic acid sequence. In some embodiments, a “modification” comprises the substitution of a “G” for an “A” in a nucleic acid sequence. In some embodiments, a “modification” comprises the substitution of an “A” for a “G” in a nucleic acid sequence. In some embodiments, a “modification” comprises the substitution of a “T” for a “C” in a nucleic acid sequence.

Several embodiments relate to compositions and methods for delivery of a recombinant DNA construct comprising an expression cassette(s) encoding a site-specific nuclease and/or any associated protein(s) to carry out genome modification. These nuclease expressing cassette(s) may be present in the same molecule or vector as a donor template for templated editing or an expression cassette comprising nucleic acid sequence encoding a genome modification enzyme as described herein (in cis) or on a separate molecule or vector (in trans). Several methods for site-directed integration are known in the art involving different sequence-specific nucleases (or complexes of proteins and/or guide RNA) that cut the genomic DNA to produce a double strand break (DSB) or nick at a desired genomic site or locus. As understood in the art, during the process of repairing the DSB or nick introduced by the nuclease enzyme, the donor template DNA, transgene, or expression cassette may become integrated into the genome at the site of the DSB or nick. The presence of the homology arm(s) in the DNA to be integrated may promote the adoption and targeting of the insertion sequence into the plant genome during the repair process through homologous recombination, although an insertion event may occur through non-homologous end joining (NHEJ). Examples of site-specific nucleases that may be used include zinc-finger nucleases, engineered or native meganucleases, TALE-endonucleases, and RNA-guided endonucleases (e.g., Cas9, CasX, CasY or Cpf1). For methods using RNA-guided site-specific nucleases (e.g., Cas9, CasX, CasY or Cpf1), the recombinant DNA construct(s) may also comprise a sequence encoding one or more guide RNAs to direct the nuclease to the desired site within the plant genome. In some embodiments, one or more guide RNAs may be provided on a separate molecule or vector (in trans).

Site-Specific Genome Modification Enzymes

Several embodiments described herein relate to compositions comprising a functionalized carbon quantum dot and a site-specific genome modification enzyme. As used herein, the term “site-specific genome modification enzyme” refers to any enzyme that can modify a nucleotide sequence in a sequence-specific manner. In some embodiments, a site-specific genome modification enzyme modifies the genome by inducing a single-strand break. In some embodiments, a site-specific genome modification enzyme modifies the genome by inducing a double-strand break. In some embodiments, a site-specific genome modification enzyme comprises a cytidine deaminase. In some embodiments, a site-specific genome modification enzyme comprises an adenine deaminase. In the present disclosure, site-specific genome modification enzymes include endonucleases, recombinases, transposases, deaminases, helicases and any combination thereof. In some embodiments, the site-specific genome modification enzyme is a sequence-specific nuclease.

In one aspect, the endonuclease is selected from a meganuclease, a zinc-finger nuclease (ZEN), a transcription activator-like effector nucleases (TALEN), an Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), an RNA-guided nuclease, such as a CRISPR associated nuclease (non-limiting examples of CRISPR associated nucleases include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1 (also known as Cas12a), CasX, CasY, homologs thereof, or modified versions thereof).

In some embodiments, the site-specific genome modification enzyme is a dCas9-Fok1 fusion protein. In another aspect, the site-specific genome modification enzyme is a dCas9-recombinase fusion protein. As used herein, a “dCas9” refers to a Cas9 endonuclease protein with one or more amino acid mutations that result in a Cas9 protein without endonuclease activity, but retaining RNA-guided site-specific DNA binding. As used herein, a “dCas9-recombinase fusion protein” is a dCas9 with a protein fused to the dCas9 in such a manner that the recombinase is catalytically active on the DNA.

In some embodiments, the site-specific genome modification enzyme is a dCas9-cytosine deaminase fusion protein. In another aspect, the site-specific genome modification enzyme is a dCas9-adenine deaminase fusion protein. In some embodiments, one or more of a dCas9-cytosine deaminase fusion protein and a dCas9-adenine deaminase fusion protein are utilized to modify a nucleic acid sequence.

In some embodiments, the site-specific genome modification enzyme is a recombinase. Non-limiting examples of recombinases include a tyrosine recombinase attached to a DNA recognition motif provided herein is selected from the group consisting of a Cre recombinase, a Gin recombinase, a Flp recombinase, and a Tnpl recombinase. In an aspect, a Cre recombinase or a Gin recombinase provided herein is tethered to a zinc-finger DNA-binding domain, or a TALE DNA-binding domain, or a Cas9 nuclease. In another aspect, a serine recombinase attached to a DNA recognition motif provided herein is selected from the group consisting of a PhiC31 integrase, an R4 integrase, and a TP-901 integrase. In another aspect, a DNA transposase attached to a DNA binding domain provided herein is selected from the group consisting of a TALE-piggyBac and TALE-Mutator.

Site-specific genome modification enzymes, such as meganucleases, ZFNs, TALENs, Argonaute proteins (non-limiting examples of Argonaute proteins include Thermus thermophilus Argonaute (TtAgo), Pyrococcus furiosus Argonaute (PfAgo), Natronobacterium gregoryi Argonaute (NgAgo), homologs thereof, or modified versions thereof), RNA-guided nucleases (non-limiting examples of RNA-guided nucleases include the CRISPR associated nucleases, such as Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1 (also known as Cas12a), CasX, CasY, homologs thereof, or modified versions thereof) and engineered RNA-guided nucleases (RGNs), induce a genome modification such as a double-stranded DNA break (DSB) or single-strand DNA break at the target site of a genomic sequence. In some embodiments, breaks or nicks in the target DNA sequence are repaired by the natural processes of homologous recombination (HR) or non-homologous end-joining (NHEJ). In some embodiments, sequence modifications occur at or near the cleaved or nicked sites, which can include deletions or insertions that result in modification of the nucleic acid sequence, or integration of exogenous nucleic acids by homologous recombination or NHEJ.

Any of the DNA of interest provided herein can be integrated into a target site of a chromosome sequence by introducing the DNA of interest and the provided site-specific genome modification enzymes. Any method provided herein can utilize any site-specific genome modification enzyme provided herein.

Several embodiments relate to a method and/or a composition provided herein comprising at least one, at least two, at least three, at least four, at least five, at least six, at least seven, at least eight, at least nine, or at least ten site-specific genome modification enzymes. In yet another aspect, a method and/or a composition provided herein comprises at least one, at least two, at least three, at least four, at least five, at least six, at least seven, at least eight, at least nine, or at least ten polynucleotides encoding at least one, at least two, at least three, at least four, at least five, at least six, at least seven, at least eight, at least nine, or at least ten site-specific genome modification enzymes.

Several embodiments relate to compositions comprising a functionalized carbon quantum dot comprising a carbon quantum dot and a a recombinase. In an aspect, a tyrosine recombinase attached to a DNA recognition motif provided herein is selected from the group consisting of a Cre recombinase, a Gin recombinase a Flp recombinase, and a Tnpl recombinase. In an aspect, a Cre recombinase or a Gin recombinase provided herein is tethered to a zinc-finger DNA binding domain. In another aspect, a serine recombinase attached to a DNA recognition motif provided herein is selected from the group consisting of a PhiC31 integrase, an R4 integrase, and a TP-901 integrase. In another aspect, a DNA transposase attached to a DNA binding domain provided herein is selected from the group consisting of a TALE-piggyBac and TALE-Mutator.

Several embodiments relate to compositions comprising a functionalized carbon quantum dot comprising a carbon quantum dot and a zinc-finger nuclease (ZFN). ZFNs are synthetic proteins consisting of an engineered zinc finger DNA-binding domain fused to the cleavage domain of the Fok1 restriction nuclease. ZFNs can be designed to cleave almost any long stretch of double-stranded DNA for modification of the zinc finger DNA-binding domain. ZFNs form dimers from monomers composed of a non-specific DNA cleavage domain of Fok1 nuclease fused to a zinc finger array engineered to bind a target DNA sequence. The DNA-binding domain of a ZFN is typically composed of 3-4 zinc-finger arrays. The amino acids at positions −1, +2, +3, and +6 relative to the start of the zinc finger ∞-helix, which contribute to site-specific binding to the target DNA, can be changed and customized to fit specific target sequences. The other amino acids form the consensus backbone to generate ZFNs with different sequence specificities. Rules for selecting target sequences for ZFNs are known in the art. The Fok1 nuclease domain requires dimerization to cleave DNA and therefore two ZFNs with their C-terminal regions are needed to bind opposite DNA strands of the cleavage site (separated by 5-7 nt). The ZFN monomer can cut the target site if the two-ZF-binding sites are palindromic. The term ZFN, as used herein, is broad and includes a monomeric ZFN that can cleave double stranded DNA without assistance from another ZFN. The term ZFN is also used to refer to one or both members of a pair of ZFNs that are engineered to work together to cleave DNA at the same site.

Without being limited by any scientific theory, because the DNA-binding specificities of zinc finger domains can in principle be re-engineered using one of various methods, customized ZFNs can theoretically be constructed to target nearly any gene sequence. Publicly available methods for engineering zinc finger domains include Context-dependent Assembly (CoDA), Oligomerized Pool Engineering (OPEN), and Modular Assembly.

Several embodiments relate to compositions comprising a functionalized carbon quantum dot comprising a carbon quantum dot and a meganuclease. Meganucleases, which are commonly identified in microbes, are unique enzymes with high activity and long recognition sequences (>14 nt) resulting in site-specific digestion of target DNA. Engineered versions of naturally occurring meganucleases typically have extended DNA recognition sequences (for example, 14 to 40 nt). The engineering of meganucleases can be more challenging than that of ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

Several embodiments relate to compositions comprising a functionalized carbon quantum dot comprising a carbon quantum dot and a transcription activator-like effector nuclease (TALEN). TALENs are artificial restriction enzymes generated by fusing the transcription activator-like effector (TALE) DNA binding domain to a nuclease domain. In one aspect, the nuclease is selected from a group consisting of PvuII, MutH, TevI and FokI, AlwI, MlyI, SbfI, SdaI, StsI, CleDORF, Clo051, Pept071. The term TALEN, as used herein, is broad and includes a monomeric TALEN that can cleave double stranded DNA without assistance from another TALEN. The term TALEN is also used to refer to one or both members of a pair of TALENs that work together to cleave DNA at the same site. Transcription activator-like effectors (TALEs) can be engineered to bind practically any DNA sequence, such as a target sequence in a nucleic acid encoding an AUX/IAA protein. TALE proteins are DNA-binding domains derived from various plant bacterial pathogens of the genus Xanthomonas. The X pathogens secrete TALEs into the host plant cell during infection. The TALE moves to the nucleus, where it recognizes and binds to a specific DNA sequence in the promoter region of a specific DNA sequence in the promoter region of a specific gene in the host genome. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

Several embodiments relate to compositions comprising a functionalized carbon quantum dot comprising a carbon quantum dot and at least one, at least two, at least three, at least four, at least five, at least six, at least seven, at least eight, at least nine, or at least ten RNA-guided nucleases. In some embodiments, a CRISPR/Cas9 system, a CRISPR/Cpf1 system, a CRISPR/CasX system, or a CRISPR/CasY system are alternatives may be used in the compositions described herein. The CRISPR systems are based on RNA-guided engineered nucleases that use complementary base pairing to recognize DNA sequences at target sites. The CRISPR (clustered regularly interspaced short palindromic repeats)/Cas (CRISPR-associated) system is an alternative to synthetic proteins whose DNA-binding domains enable them to modify genomic DNA at specific sequences (e.g., ZFN and TALEN). CRISPR/Cas systems are part of the adaptive immune system of bacteria and archaea, protecting them against invading nucleic acids such as viruses by cleaving the foreign DNA in a sequence-dependent manner. The immunity is acquired by the integration of short fragments of the invading DNA known as spacers between two adjacent repeats at the proximal end of a CRISPR locus. The CRISPR arrays, including the spacers, are transcribed during subsequent encounters with invasive DNA and are processed into small interfering CRISPR RNAs (crRNAs) approximately 40 nt in length, which combine with the trans-activating CRISPR RNA (tracrRNA) to activate and guide the Cas9 nuclease. This cleaves homologous double-stranded DNA sequences known as protospacers in the invading DNA. A prerequisite for cleavage is the presence of a conserved protospacer-adjacent motif (PAM) downstream of the target DNA, which usually has the sequence 5′-NGG-3′ but less frequently NAG. Specificity is provided by the so-called “seed sequence” approximately 12 bases upstream of the PAM, which must match between the RNA and target DNA. Cpf1 acts in a similar manner to Cas9, but Cpf1 does not require a tracrRNA. Specificity of the CRISPR/Cas system is based on an RNA-guide that use complementary base pairing to recognize target DNA sequences.

Several embodiments relate to compositions comprising a functionalized carbon quantum dot and a RNA-guided Cas nuclease (non-limiting examples of RNA-guided nucleases include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, or modified versions thereof); and, optionally, the guide RNA necessary for targeting the respective nucleases.

In one aspect, a method and/or composition provided herein comprises one or more, two or more, three or more, four or more, five or more, six or more, seven or more, eight or more, nine or more, or ten or more RNA-guided nucleases in combination with a CRISPR guide RNA (e.g., crRNA and/or tracrRNA). In one aspect, a method and/or composition provided herein comprises vectors comprising polynucleotides encoding one or more, two or more, three or more, four or more, five or more, six or more, seven or more, eight or more, nine or more, or ten or more RNA-guided nucleases. Preferably, vectors comprising polynucleotides encoding one or more, two or more, three or more, four or more, five or more, six or more, seven or more, eight or more, nine or more, or ten or more RNA-guided nucleases are provided to a cell by the functionalized carbon dots provided herein.

Several embodiments relate to plant cells, plant tissue, plant seed and plants produced by the methods disclosed herein. Plants may be monocots or dicots, and may include, for example, rice, wheat, barley, oats, rye, Sorghum, maize, grapes, tomatoes, potatoes, lettuce, broccoli, cucumber, peanut, melon, leeks, onion, soybean, alfalfa, sunflower, cotton, canola, and sugar beet plants.

Methods of Making Polynucleotides

Methods of making polynucleotides are well known in the art. Chemical synthesis, in vivo synthesis and in vitro enzymatic synthesis methods and compositions are known in the art and include various viral elements, microbial cells, modified polymerases, and modified nucleotides. Commercial preparation of oligonucleotides often provides two deoxyribonucleotides on the 3′ end of the sense strand. Long polynucleotide molecules can be synthesized from commercially available kits, for example, kits from Applied Biosystems/Ambion (Austin, Tex.) have DNA ligated on the 5′ end in a microbial expression cassette that includes a bacterial T7 polymerase promoter that makes RNA strands that can be assembled into a dsRNA and kits provided by various manufacturers that include T7 RiboMax Express (Promega, Madison, Wis.), AmpliScribe T7-Flash (Epicentre, Madison, Wis.), and TranscriptAid T7 High Yield (Fermentas, Glen Burnie, Md.). Polynucleotides as described herein can be produced from microbial expression cassettes in bacterial cells (Ongvarrasopone et al. ScienceAsia 33:35-39; Yin, Appl. Microbiol. Biotechnol 84:323-333, 2009; Liu et al., BMC Biotechnology 10: 85, 2010). In some embodiments, the bacterial cells have regulated or deficient RNase III enzyme activity. In some embodiments, fragments of target genes are inserted into the microbial expression cassettes in a position in which the fragments are express to produce ssRNA or dsRNA useful in the methods described herein to regulate expression of the target gene. Long polynucleotide molecules can also be assembled from multiple RNA or DNA fragments. In some embodiments, design parameters such as Reynolds score (Reynolds et al. Nature Biotechnology 22, 326-330 (2004) and Tuschl rules (Pei and Tuschl, Nature Methods 3(9): 670-676, 2006) are known in the art and are used in selecting polynucleotide sequences effective in gene silencing. In some embodiments, random design or empirical selection of polynucleotide sequences is used in selecting polynucleotide sequences effective in gene silencing. In some embodiments, the sequence of a polynucleotide is screened against the genomic DNA of the intended plant to minimize unintentional silencing of other genes.

Methods for in vitro and in vivo expression of RNA for large scale production are known in the art. For example, methods for improved production of dsRNA are disclosed in WO 2014/151581.

Following synthesis or production, the polynucleotides may optionally be purified. For example, polynucleotides can be purified from a mixture by extraction with a solvent or resin, precipitation, electrophoresis, chromatography, or a combination thereof. Alternatively, polynucleotides may be used with no, or a minimum of, purification to avoid losses due to sample processing. The polynucleotides may be dried for storage or dissolved in an aqueous solution. The solution may contain buffers or salts to promote annealing, and/or stabilization of the duplex strands.

Other Compositions

Other compositions of the present invention include various dispersion compositions (e.g., agrochemical formulations). In general, these compositions comprise the particulate composition as described herein and a liquid medium (e.g., solvent) such as water.

The dispersion compositions can comprise a plurality of the particulate compositions dispersed in a liquid medium. In these embodiments, the plurality of particulates can be characterized by an average particle size. Average particle size (i.e., average particle diameter) can be measured by dynamic light scattering (DLS), transmission electron microscopy (TEM), atomic force microscopy (AFM), or size exclusion chromatography (SEC). Preferably, the average particle size is measured by dynamic light scattering (DLS) or size exclusion chromatography (SEC). In various embodiments, the plurality of particulates can have an average particle size that is no greater than about 21 nm, no greater than about 18 nm, no greater than about 15 nm, no greater than about 12 nm, or no greater than about 10 nm. For example, the plurality of particulates can have an average particle size that is from about 0.5 nm to about 21 nm, from about 0.5 nm to about 18 nm, from about 0.5 nm to about 15 nm, from about 0.5 nm to about 12 nm, from about 0.5 nm to about 10 nm, from about 0.5 nm to about 8 nm, from about 1 nm to about 21 nm, from about 1 nm to about 18 nm, from about 1 nm to about 15 nm, from about 1 nm to about 12 nm, from about 1 nm to about 10 nm, from about 1 nm to about 8 nm, from about 5 nm to about 21 nm, from about 5 nm to about 18 nm, from about 5 nm to about 15 nm, from about 5 nm to about 12 nm, from about 5 nm to about 10 nm, or from about 5 nm to about 8 nm.

Various dispersion compositions of the present invention comprise the particulate composition, as described herein, or plurality thereof, a surfactant, and a solvent.

In some embodiments, the surfactant comprises a nonionic surfactant. For example, the surfactant can include at least one nonionic surfactant selected from the group consisting of organosilicone surfactants, alkoxylated fatty acids and alcohols, alkoxylated sorbitan esters, alkylpolyglucosides, PEO-PPO block copolymers, glycerides, and combinations thereof.

In some embodiments, dispersion compositions of the present invention comprise one or more agents for conditioning the surface of a plant to permeation by the macromolecules and macromolecular complexes described herein. Agents for conditioning the surface of a plant to permeation include surfactants, organic solvents, aqueous solutions or aqueous mixtures of organic solvents, oxidizing agents, acids, bases, oils, enzymes, or combinations thereof. Examples of useful surfactants include sodium or lithium salts of fatty acids (such as tallow or tallowamines or phospholipids) and organosilicone surfactants. Other useful surfactants include organosilicone surfactants including nonionic organosilicone surfactants, e. g., trisiloxane ethoxylate surfactants or a silicone polyether copolymer such as a copolymer of polyalkylene oxide modified heptamethyl trisiloxane and allyloxypolypropylene glycol methylether (commercially available as Silwet® L-77 surfactant having CAS Number 27306-78-1 and EPA Number: CAL. REG. NO. 5905-50073-AA, currently available from Momentive Performance Materials, Albany, N.Y.). When Silwet L-77 surfactant is used as a pre-spray treatment of plant leaves or other surfaces, concentrations in the range of about 0.015 to about 2 percent by weight (wt %) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt %) are efficacious in preparing a leaf or other plant surface for transfer of polynucleotide molecules into plant cells from a topical application on the surface.

The dispersion compositions can be application mixtures that are suitable for applying to plants or concentrate compositions that are convenient for storage and transport, but typically require dilution with water or additional solvent before use. In various dispersion compositions, the concentration of the polynucleotide can be at least about 0.00001 wt. %, at least about 0.0001 wt. %, at least about 0.0005 wt. %, or at least about 0.001 wt. %. Also, in some embodiments, the concentration of the surfactant can at least about 0.001 wt. %, at least about 0.005 wt. %, at least about 0.01 wt. %, at least about 0.05 wt. %, at least about 0.1 wt. %, at least about 0.5 wt. %, at least about 1 wt. %, or at least about 2 wt. %.

In various embodiments wherein the dispersion composition is an application mixture, the concentration of the polynucleotide and/or protein can be from about 0.00001 wt. % to about 1 wt. %, from about 0.00001 wt. % to about 0.1 wt. %, from about 0.00001 wt. % to about 0.01 wt. %, from about 0.00001 wt. % to about 0.001 wt. %, from about 0.00001 wt. % to about 0.0001 wt. %, from about 0.00005 wt. % to about 1 wt. %, from about 0.00005 wt. % to about 0.1 wt. %, from about 0.00005 wt. % to about 0.01 wt. %, from about 0.00005 wt. % to about 0.001 wt. %, from about 0.00005 wt. % to about 0.0001 wt. %, from about 0.0001 wt. % to about 1 wt. %, from about 0.0001 wt. % to about 0.1 wt. %, from about 0.0001 wt. % to about 0.01 wt. %, from about 0.0001 wt. % to about 0.001 wt. %, from about 0.0005 wt. % to about 1 wt. %, from about 0.0005 wt. % to about 0.1 wt. %, from about 0.0005 wt. % to about 0.01 wt. %, or from about 0.0005 wt. % to about 0.001 wt. %. In these and other embodiments, the concentration of the surfactant can be from about 0.001 wt. % to about 1 wt. %, from about 0.001 wt. % to about 0.5 wt. %, from about 0.001 wt. % to about 0.1 wt. %, from about 0.001 wt. % to about 0.05 wt. %, from about 0.01 wt. % to about 1 wt. %, from about 0.01 wt. % to about 0.5 wt. %, from about 0.01 wt. % to about 0.1 wt. %, or from about 0.01 wt. % to about 0.05 wt. %.

In various embodiments wherein the dispersion composition is a concentrate compositions, the concentration of the polynucleotide and/or protein is from about 0.0001 wt. % to about 1 wt. %, from about 0.0001 wt. % to about 0.1 wt. %, from about 0.0001 wt. % to about 0.01 wt. %, from about 0.0001 wt. % to about 0.001 wt. %, from about 0.0005 wt. % to about 1 wt. %, from about 0.0005 wt. % to about 0.1 wt. %, from about 0.0005 wt. % to about 0.01 wt. %, from about 0.0005 wt. % to about 0.001 wt. %, from about 0.001 wt. % to about 1 wt. %, from about 0.001 wt. % to about 0.1 wt. %, from about 0.001 wt. % to about 0.01 wt. %, from about 0.005 wt. % to about 1 wt. %, from about 0.005 wt. % to about 0.1 wt. %, or from about 0.005 wt. % to about 0.01 wt. %. In these and other embodiments, the concentration of the surfactant can be from about 0.01 wt. % to about 10 wt. %, from about 0.01 wt. % to about 5 wt. %, from about 0.01 wt. % to about 1 wt. %, from about 0.01 wt. % to about 0.5 wt. %, from about 0.1 wt. % to about 10 wt. %, from about 0.1 wt. % to about 5 wt. %, from about 0.1 wt. % to about 1 wt. %, from about 0.1 wt. % to about 0.5 wt. %, from about 0.5 wt. % to about 10 wt. %, from about 0.5 wt. % to about 5 wt. %, or from about 0.5 wt. % to about 1 wt. %.

In some embodiments, the dispersion compositions can further comprise an osmoticum (also referred to as an osmolyte). An osmoticum is a compound that affects osmosis. Examples of osmoticums include sucrose, mannitol, fructose, galactose, sodium chloride, glycerol, sorbitol, polyalchohols, proline, trehalose, trimethylamine N-oxide (TMAO), dimethyl sulfoniopropionate, trimethylglycine, sarcosine, betaine, glycerophosphorylcholine, myo-inositol, taurine, and glycine. In certain embodiments, the osmoticum is selected from the group consisting of sucrose, mannitol, glycerol, and combinations thereof.

However, in other embodiments, the dispersion compositions can be essentially free or free of an osmoticum.

In some embodiments, the dispersion compositions can further comprise one or more additional agrochemicals. Additional agrochemicals include various fertilizers and pesticides (e.g., insecticides, fungicides, herbicides, and nematicides).

### II. Methods of Use

The present invention is also directed to various methods for delivering a macromolecule or macromolecular complex into a plant cell. In some embodiments, the macromolecule is a polynucleotide. In some embodiments, the macromolecule is a protein. In some embodiments, the macromolecular complex is a ribonucleoprotein. In general, these methods comprise applying a dispersion composition as described herein onto a plant and/or a part thereof.

The dispersion compositions can be applied to a variety of plant species. Plants that are particularly useful in the methods of the present invention include all plants which belong to the super family Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp., Dolichos spp., Dorycnium rectum, Echinochloa pyramidalis, Ehraffia spp., Eleusine coracana, Eragrestis spp., Erythrina spp., Eucalyptus spp., Euclea schimperi, Eulalia villosa, Pagopyrum spp., Feijoa sellowlana, Fragaria spp., Flemingia spp., Freycinetia banksli, Geranium thunbergii, Ginkgo biloba, Glycine javanica, Gliricidia spp., Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, sugar beet, sugar cane, sunflower, tomato, squash tea, maize, wheat, barley, rye, oat, peanut, pea, lentil and alfalfa, cotton, rapeseed, canola, pepper, sunflower, tobacco, eggplant, Eucalyptus, a tree, an ornamental plant, a perennial grass and a forage crop.

In some embodiments, the plant comprises a crop plant including, but not limited to, cotton, Brassica vegetables, oilseed rape, sesame, olive tree, oil palm, banana, wheat, corn or maize, barley, alfalfa, peanuts, sunflowers, rice, oats, sugarcane, soybean, turf grasses, barley, rye, Sorghum, sugar cane, chicory, lettuce, tomato, zucchini, bell pepper, eggplant, cucumber, melon, watermelon, beans, hibiscus, okra, apple, rose, strawberry, chili, garlic, pea, lentil, canola, mums, Arabidopsis, broccoli, cabbage, beet, quinoa, spinach, squash, onion, leek, tobacco, potato, sugar beet, papaya, pineapple, mango, Arabidopsis thaliana, and also plants used in horticulture, floriculture or forestry, such as, but not limited to, poplar, firs, Eucalyptus, pine, ornamental plants, perennial grasses, and coniferous plants.

The methods of the present invention are also suitable for use with algae and other non-Viridiplantae.

### III. Processes for Preparing Compositions

The present invention is also directed to various processes for preparing the various compositions described herein.

As noted herein, carbon quantum dots can be synthesized by various techniques including the “top down” and “bottom-up” approaches. In various embodiments, the carbon quantum dots are prepared by the bottom-up approach. In this technique, a carbon quantum dot precursor compound, as described herein, are heated at elevated temperature such as from about 75° C. to about 300° C., from about 75° C. to about 200° C., from about 100° C. to about 300° C. or from about 100° C. to about 200° C. to carbonize the precursor thereby forming the carbon quantum dot. Heating can be conducted by various means. For example, heating can be conducted via microwave or autoclave.

In various embodiments, the carbon quantum dot precursor compound is mixed with a solvent. Solvents include, for example, water, organic solvents, or mixtures of water and organic solvents. Organic solvents could also include chlorinated solvents (e.g., chloroform).

Also noted herein, various processes can be used to prepare the functionalized carbon quantum dots. Some processes comprise mixing a carbon quantum dot precursor compound and a cationic polymer to form a precursor mixture and carbonizing the carbon quantum dot precursor compound to form functionalized carbon quantum dots. In other processes, the carbon quantum dot is formed first and then functionalized. These processes comprise carbonizing a carbon quantum dot precursor compound as described herein to form carbon quantum dots and mixing the carbon quantum dots with a cationic polymer to form the functionalized carbon quantum dots.

After forming the functionalized carbon quantum dot, the polynucleotide can be complexed with the functionalized carbon quantum dot to form a particulate composition. For example, in some embodiments, processes for preparing a particulate composition comprise mixing a carbon quantum dot precursor compound and a cationic polymer (e.g., a cationic polymer comprising one or more amine functional groups and having an average molecular weight of from about 3 kDa to about 15 kDa) to form a precursor mixture; carbonizing the carbon quantum dot precursor compound to form functionalized carbon quantum dots; and complexing one or more polynucleotides for regulating or modulating of a gene expression in a plant cell with the functionalized carbon quantum dots to form the particulate composition (e.g., wherein at least a portion of the functionalized carbon quantum dots have a particle size that is no greater than about 15 nm, no greater than about 12 nm, or no greater than about 10 nm). In certain embodiments, processes for preparing a particulate composition comprise carbonizing a carbon quantum dot precursor compound to form carbon quantum dots; mixing the carbon quantum dots with a cationic polymer (e.g., a cationic polymer comprising one or more amine functional groups and having an average molecular weight of from about 3 kDa to about 15 kDa) to form functionalized carbon quantum dots; complexing one or more polynucleotides for regulating or modulating of a gene expression in a plant cell with the functionalized carbon quantum dots to form the particulate composition (e.g., wherein at least a portion of the functionalized carbon quantum dots have a particle size that is no greater than about 15 nm, no greater than about 12 nm, or no greater than about 10 nm).

After synthesis, the carbon quantum dots or functionalized carbon quantum dots can be separated from uncarbonized carbon quantum dot precursor compound and by-products of carbonization. For example, the carbon quantum dots or functionalized carbon quantum dots can be purified or fractionated by ultrafiltration, dialysis, size exclusion chromatography, and combinations thereof to remove unreacted precursors and by-products. In some embodiments, the processes further comprise fractionating the carbon quantum dots (or functionalized carbon quantum dots) to form two or more fractions of carbon quantum dots having different particle size distributions. In various embodiments, at least about 70%, at least about 80%, at least about 90%, or at least about 95% of the functionalized carbon quantum dots have a particle size that is no greater than about 15 nm, no greater than about 12 nm, or no greater than about 10 nm.

Further, the dispersion compositions of the present invention can be prepared by mixing the particulate composition as described herein with solvent and other ingredients such as one or more surfactants.

### EXAMPLES

The following non-limiting examples are provided to further illustrate the present invention.

### Example 1: Synthesis of Carbon Quantum Dots Using Microwave Pyrolysis

Polyethylene glycol (PEG) with an average molecular weight of 200 Da (MW 200 Da) (400 mg) and branched polyethyleneimine (bPEI) with an average molecular weight of 10,000 Da (MW 10,000 Da) (350 mg) were added to 10 mL of 0.1 N aqueous HCl in a 125 mL Erlenmeyer flask. The mixture was stirred continuously for approximately 60 minutes followed by degassing under vacuum. One gram of Teflon boiling stones were added to the flask and the resulting solution was heated in a 700 W microwave on high power for approximately 2.5 to 3.5 minutes. The formation of bPEI functionalized carbon quantum dots occurred shortly after evaporation of the liquid. The preparation had a light yellowish color and showed blue fluorescence under UV light.

### Example 2: Synthesis of Carbon Quantum Dots Using Chloroform Reflux

PEG (MW 200 Da) (400 mg) and bPEI (MW 10,000 Da) (350 mg) were added to 10 mL of chloroform (CHCl₃). One gram of Teflon boiling stones was added to the solution and allowed to reflux for 1.5 hours. After cooling to room temperature, the chloroform was dried under nitrogen.

### Example 3: Synthesis of Carbon Quantum Dots Using Autoclave Formation

Glycerol (400 mg) and bPEI (MW 10,000 Da) (350 mg) were added to 10 mL of water. The pH was adjusted to 8.0 and the resulting solution was autoclaved for 2 hours and 45 minutes at 121° C., 100 kPA (15 PSI) to form bPEI functionalized carbon quantum dots.

### Example 4: Purification of Carbon Quantum Dots

Purification of the preparations of Examples 1 to 3 was performed to remove precursors and by-products, which could decrease delivery efficiency. The carbon quantum dot preparations of Examples 1-3 were loaded on a Sephacryl S-300 HR or Sephadex G50 size exclusion column equilibrated with 10 mM NaCl. The column was eluted with 10 mM NaCl and absorbance was monitored at 360 nm.

### Example 5: Formulation of Functionalized Carbon Quantum Dots with dsRNA

Functionalized carbon dots prepared as described in Examples 1 to 3 were formulated with dsRNA for plant delivery. Preparations for plant delivery were in 2-(N-morpholino)ethanesulfonic acid (MES) buffer pH 5.7 to 6.2 to a final concentration of 10 mM. The concentration of dsRNA used was determined as follows.

Functionalized carbon quantum dots used for plant delivery have an absorption max around 360 nm. The extinction coefficient of the carbon quantum dots is not known, but the relative concentrations of different preparations or purified fractions can be determined by measuring absorption at 360 nm. For each microgram (μg) of dsRNA used, approximately 2.5 μL of a colloidal solution with ABS360 of 1.0 was used following this equation:

Volume of CDOTs (μL)=μg RNA*1/ABS360*2.5

Generally, the RNA and the carbon dots were prepared in separate aliquots of the same IVIES buffer before combining and mixing via vortexing or stirring. The combined buffer containing RNA and the carbon dots was then incubated for about one hour at room temperature to allow for complexation. Formulations were stable for at least 48 h at room temperature, 37° C., or 45° C. Formulations are stable at 4° C. for at least several months. The RNA used in the Examples herein was either chemically synthesized from Integrated DNA Technologies or produced using methods known in the art.

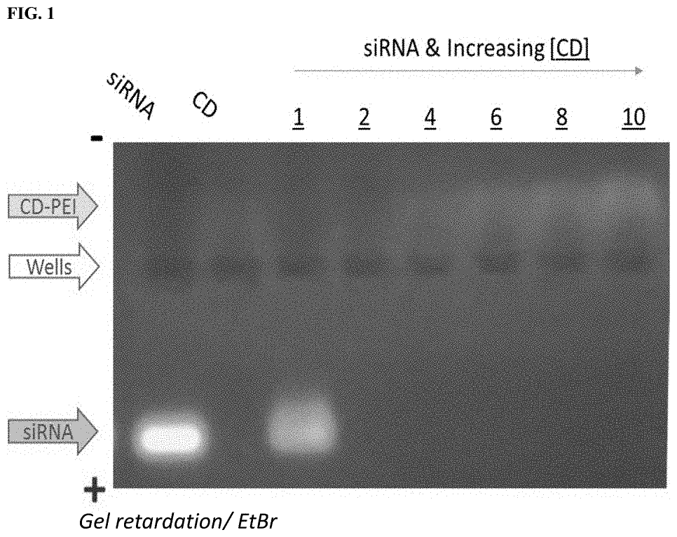

The ability of the functionalized carbon dots to bind siRNA or longer dsRNA molecules was tested by gel retardation assays (FIG. 1). Encapsulation of the dsRNA results in reduced binding of ethidium bromide and/or failure to migrate in the gel.

### Example 6: Treatment of Formulated Functionalized Carbon Quantum Dots-dsRNA Complexes with RNase Confirmed Stability of the Complex

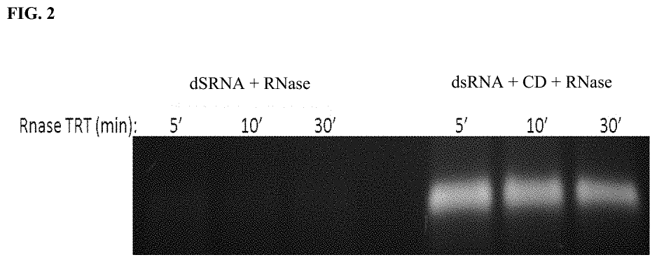

dsRNA formulated with and without functionalized carbon quantum dots was treated with E. coli RNase III for 5 to 30 minutes. The reaction buffer contained 20 nM Tris-Cl pH 8.0, 0.5 mM EDTA, 5 mM MgCl₂, 1 mM DTT, 140 mM NaCl, 2.7 mM KCl. Reactions containing 160 ng of RNA and 0.06 μg of RNase III in a total volume of 20 μL were incubated for 5, 10, or 30 minutes at room temperature. Following the incubation period, SDS was added to a final concentration of 1% to dissociate the bound dsRNA from the carbon quantum dots. The stability of the dsRNA was then monitored by agarose gel electrophoresis with ethidium bromide staining. dsRNA formulated with functionalized carbon quantum dot was visible in the agarose gel, demonstrating its stability up to 30 minutes in the presence of an RNase. dsRNA formulated without carbon quantum dot were degraded and failed to show on the gel (FIG. 2).

### Example 7: Transfection of Functionalized Carbon Quantum Dot-dsRNA Complexes into Tobacco BY-2 Suspension Cells

To test the ability of functionalized carbon quantum dots to deliver dsRNA into cells, silencing assays were performed with a stably transformed dual luciferase reporter line of BY-2 cells. The firefly luciferase was targeted for silencing and a Renilla luciferase was used for normalization. Functionalized carbon quantum dots were synthesized using the microwave pyrolysis method outlined in Example 1 using PEG, or glycerol as the carbon quantum dot precursor compound and bPEI (MW 1800 Da) as the functionalizing cationic polymer. Additional functionalized carbon quantum dots were synthesized using the autoclave method outlined in Example 3 (modified by autoclaving for one hour) using citrate as the carbon quantum dot precursor compound and bPEI (MW 1800 Da) as the functionalizing cationic polymer. The carbon dots were then formulated with dsRNA and transfected into a stably transformed tobacco BY-2 cell line expressing a Renilla luciferase and a Firefly luciferase reporter gene. Two dsRNAs were delivered: a non-target 24 blunt end control dsRNA comprising a sequence as set forth in SEQ ID NO:2 and a 21-mer dsRNA directed against the Firefly luciferase gene comprising a sequence as set forth in SEQ ID NO:1 (Table 1). A low dose of dsRNA (0.016 mg/mL) was used. All formulations (incubation buffer) were prepared as described in Example 4 in a 10 mM MES buffer (pH 5.7) and also contained 100 mM sucrose. Cells were treated for 1 hour and then washed 2× with W5 buffer and once in incubation buffer. After treatments, cells were incubated for 16 hours and the activity of the two luciferase reporters were measured with a PROMEGA dual luciferase assay kit. Renilla luciferase activity was then used to normalize for differences in cell number. Knockdown was calculated as the difference in normalized firefly luciferase activity between the formulation control (SEQ ID NO:2) and the formulated firefly luciferase siRNA (SEQ ID NO:1). Table 2 summarizes the percent knock down observed in the different formulations used.

TABLE 1
dsRNA Sequences for Luciferase Assays

| | | SEQ |
|---|---|---|
| Gene Target | SEQUENCE | ID NO: |
| Firefly | GAUAUGGGCUGAAUACAAAUC: | 1 |
| Luciferase | UUUGUAUUCAGCCCAUAUCGU | |
| non-target | AUGCCAGAUGUUGCUAUGACUCUU: | 2 |
| 24 blunt end | AAGAGUCAUAGCAACAUCUGGCAU | |

TABLE 2
Gene specific knock down of Firefly luciferase activity
in BY-2 suspension cells
measured after transfection with dsRNA targeting
the luciferase gene that was complexed
with carbon quantum dots.

| | Percent knock down in |
|---|---|
| | Firefly luciferase activity |
| Carbon quantum dot formulation | (SEQ ID NO: 1) |
| Citrate-Trp-bPEI-I (MW 1800 Da) | 17% |
| Citrate-bPEI-II(MW 1800 Da) | 26% |
| PEG-bPEI-II(MW 1800 Da) | 49% |
| Glycerol-bPEI (MW 1800 Da) | 54% |

This experiment demonstrated that the functionalized carbon quantum dots produced using glycerol and PEI had better efficacy in suppressing Firefly luciferase expression than carbon dots derived from citrate.

### Example 8: Carbon Quantum Dots Functionalized with Branched PEI (MW 10,000 Da) Provided for Enhanced Efficiency at Delivering dsRNA and Achieving Silencing

Functionalized carbon quantum dot complexed with dsRNA were also tested for dsRNA delivery and silencing efficacy in whole plants using a GFP expression line of tomato. Under blue lights chlorophyll has a strong red fluorescence that can be masked by the expression of a GFP transgene. Silencing of GFP is easily detected by the un-masking of the chlorophyll fluorescence.

Functionalized carbon quantum dots were prepared using the microwave pyrolysis methodology of Example 1 using either PEG with bPEI (MW 1800 Da) or bPEI (MW 10,000 Da). Following purification and formulation with a 22-mer dsRNA (0.01 mg/mL) targeting GFP or a nonspecific 22-mer dsRNA, the formulations were applied to tomato plants that constitutively express GFP to determine if silencing would take place. Sequences for the dsRNAs used are provided in Table 3. Six leaves per plant received the application. All formulations were prepared as described in Example 4 in a 10 mM IVIES buffer (pH 5.7) and also contained 100 mM sucrose and 0.4% Silwet L-77 to facilitate stomatal flooding. GFP silencing was quantified by determining the area in each leaf where chlorophyll fluorescence was detected. Table 4 summarizes the % GFP silencing achieved in each condition.

TABLE 3
dsRNA sequences for GFP targeting

| dsRNA Target | Sequence | SEQ ID NO: |
|---|---|---|
| GFP 22-mer | GGCAUCAAGGUGAACUUCAAAA: | 3 |
| | UUGAAGUUCACCUUGAUGCCGU | |
| non-specific | GAUAUGGGCUGAAUACAAAUC: | 4 |
| 22-mer | UUUGUAUUCAGCCCAUAUCGU | |

TABLE 4
Percent (%) silenced area in tomato leaves after application with
functionalized carbon quantum dots formulated with dsRNA

| Carbon quantum dot | | % GFP |
|---|---|---|
| formulation | dsRNA Target | silencing |
| PEG-bPEI (MW 1800 Da) | GFP 22-mer | 20.04% |
| PEG-bPEI (MW 10,000 Da) | GFP 22-mer | 46.44% |
| PEG-bPEI (MW 10,000 Da) | Nonspecific 22-mer | 1.49% |

The p-value for the percent GFP silencing in the PEG-PEI delivery applications was 0.0012. This example indicated that the carbon quantum dot formulations with PEG-bPEI (MW 10,000 Da) was more effective at delivering the GFP dsRNA and achieving silencing throughout the leaves.

### Example 9: Functionalized Carbon Quantum Dot-dsRNA Delivery in Tomato Plants in the Absence of Sucrose as Osmoticum

In this example, a formulation of functionalized carbon quantum dots PEG-bPEI (MW 10,000 Da) prepared using the microwave pyrolysis methodology outlined in Example 1 was formulated with a 22-mer GFP targeting dsRNA at the dosage of 0.01 mg/mL and applied without sucrose to tomato plants that constitutively express the GFP gene. Formulations lacking sucrose were prepared with 10 mM MES buffer with 0.4% Silwet L77. Six leaves per plant were treated with the formulation. Table 5 summarizes the percent silencing achieved in this experiment. As in Example 8, % silencing was determined by measuring the area of the leaves showing loss of GFP fluorescence.

TABLE 5
Percent silenced area in tomato leaves
after carbon quantum dot-dsRNA
application without sucrose

| Carbon quantum | | % GFP |
|---|---|---|
| dot formulation | dsRNA Target | silencing |
| PEG-bPEI (MW 10,000 Da) | GFP 22-mer | 38% |
| PEG-bPEI (MW 10,000 Da) | Nonspecific trigger, 22-mer | 1.6% |

The p-value for this experiment was 0.0003, suggesting that carbon quantum dot delivery of dsRNA can be effective without an osmoticum (e.g., sucrose).

### Example 10: Comparison of Carbon Quantum Dot Delivery Characteristics in BY-2 Suspension Cells or Tomato Plants

A comparison of delivery efficacy in BY-2 suspension cells or in tomato plants is summarized below in Table 6. The preparation method for each functionalized carbon dots is also indicated. Each application was performed as described in Example 7 (BY-2 cells) or in Example 8 (Tomato plants) and delivery efficacy was measured as described therein. For each experiment, the functionalized carbon quantum dots were formulated with 0.01 mg/mL dsRNA targeting either the firefly luciferase gene (BY-2 cells) or the GFP gene (Tomato). Efficacy was determined by loss of luciferase fluorescence in BY-2 cells or increased chlorophyll fluorescence in tomato leaves. The relative efficacy of each formulation ranged from no efficacy (−), to low, medium and high efficacy (+, ++, +++).

TABLE 6
Summary of the carbon quantum
dots and their efficacy in BY-2 cells or tomato
plants.

| | Cationic | Efficacy in | Efficacy in | Preparation |
|---|---|---|---|---|
| Precursor | polymer | BY-2 cells | Tomato | method |
| Citrate | bPEI | + | − | Autoclave |
| | (MW 1800 Da) | | | (Example 3) |
| PEG | bPEI | ++ | + | Microwave |
| | (MW 1800 Da) | | | (Example 1) |
| Glycerol | bPEI | ++ | + | Autoclave |
| | (MW 1800 Da) | | | (Example 3) |
| PEG | PDDA | ++ | + | Microwave |
| | | | | (Example 1) |
| PEG | bPEI | +++ | ++ | Microwave |
| | (MW 10,000 Da) | | | (Example 1) |
| Glycerol | bPEI | +++ | ++ | Autoclave |
| | (MW 10,000 Da) | | | (Example 3) |
| Citrate | bPEI | N/A | − | Autoclave |
| | (MW 10,000 Da) | | | (Example 3) |

These results indicated that the PEG-bPEI or Glycerol-bPEI (MW 10,000 Da) provided for enhanced delivery of dsRNA in both BY-2 suspension cells or tomato plants relative to the carbon dots produced from citrate.

### Example 11: A Reduction in RNA and Protein Levels was Observed in Tomato Plants Treated with Functionalized Carbon Quantum Dots Formulated with dsRNA

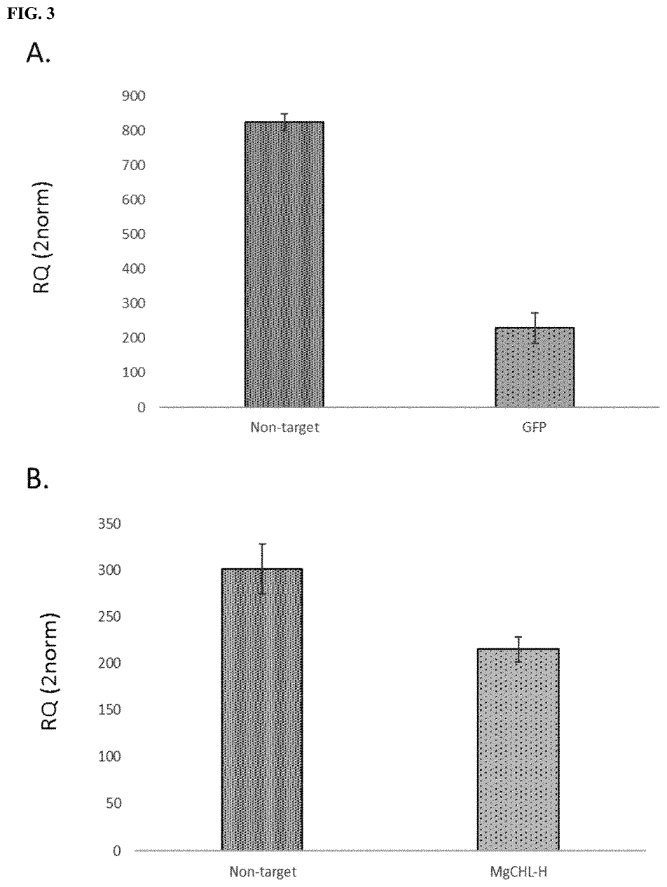

In this example, a formulation of functionalized carbon quantum dots PEG-bPEI (MW 10,000 Da) prepared using the microwave pyrolysis methodology outlined in Example 1 was formulated with a 22-mer GFP targeting dsRNA (SEQ ID NO: 3) at the dosage of 0.01 mg/mL and applied in the absence of sucrose to tomato plants that constitutively express the GFP gene. In a separate experiment, dsRNA targeting the Magnesium Chelatase (MgChl; SEQ ID NO 5: GAATGTCTTTGCTTCCATATTT:GTATGGAAGCAAAGACATTCAA) was formulated with functionalized carbon quantum dots PEG-bPEI (MW 10,000 Da) in the absence of sucrose. In each experiment, six leaves per plant were treated with the formulations. Leaves were harvested at two days after treatment for Northern analysis, three days after treatment for quantitative RT-PCR analysis and five days after treatment for Western blot analysis. For the leaves treated with carbon dots complexed with dsRNA targeting MgChl, the analysis performed was quantitative RT PCR. The results for the Northern blot analysis revealed a 38% decrease in GFP mRNA message (p-value=0.0003) relative to a non-specific control. Similarly, Western blot analysis showed a 30% reduction in GFP protein relative to a non-specific control when the relative band intensity was quantitated. The results of the quantitative RT-PCR analysis revealed a 72% reduction in GFP message and a 29% reduction in MgChl RNA levels. The quantitative RT-PCR results are summarized in FIG. 3.

Having described the invention in detail, it will be apparent that modifications and variations are possible without departing from the scope of the invention defined in the appended claims.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions, methods and processes without departing from the scope of the invention, it is intended that all matter contained in the above description and shown in the accompanying figures shall be interpreted as illustrative and not in a limiting sense.

1. A particulate composition comprising:
a functionalized carbon quantum dot comprising a carbon quantum dot and a cationic polymer comprising one or more amine functional groups, wherein the cationic polymer has an average molecular weight of from about 1 kDa to about 15 kDa; and
a polynucleotide for regulating or modulating the expression of a gene or for the expression of a non-native protein in a plant cell that is complexed with the functionalized carbon quantum dot, wherein the functionalized carbon quantum dot has a particle size that is no greater than about 15 nm.

2. The particulate composition of claim 1 wherein the cationic polymer has an average molecular weight of from about 4 kDa to about 12 kDa.

3. The particulate composition of claim 1 wherein the cationic polymer comprises a polyethyleneimine.

4. The particulate composition of claim 1 wherein the cationic polymer comprises a branched polyethyleneimine.

5. The particulate composition of claim 1 wherein the cationic polymer comprises a polydiallyldimethylammonium polymer.

6. The particulate composition of claim 1 wherein the cationic polymer comprises a mixture of two or more polymers having different average molecular weights.

7. The particulate composition of claim 1 wherein the functionalized carbon quantum dot has a particle size that is no greater than about 12 nm.

8. The particulate composition of claim 1 wherein the functionalized carbon quantum dot has a particle size that is from about 0.5 nm to about 15 nm.

9. The particulate composition of claim 1 wherein the carbon quantum dot comprises a carbonization product of at least one carbon quantum dot precursor compound selected from the group consisting of a polyol, a saccharide, a saccharide derivative, and combinations thereof.

10. (canceled)

11. The particulate composition of claim 1 wherein the carbon quantum dot comprises a carbonization product of at least one carbon quantum dot precursor compound comprising polyethylene glycol having an average molecular weight of from about 100 Da to about 500 Da.

12. (canceled)

13. (canceled)

14. The particulate composition of claim 1 wherein the polynucleotide is selected from the group consisting of single-stranded DNA (ssDNA), single-stranded RNA (ssRNA), double-stranded DNA (dsDNA), double-stranded RNA (dsRNA), and RNA/DNA hybrid.

15. (canceled)

16. (canceled)

17. The particulate composition of claim 1 wherein the polynucleotide is a small interfering RNA (siRNA).

18. A dispersion composition comprising:
the particulate composition of claim 1, or a plurality thereof;
a surfactant; and
a solvent.

19. (canceled)

20. The dispersion composition of claim 18 wherein the surfactant comprises at least one nonionic surfactant selected from the group consisting of organosilicone surfactants, alkoxylated fatty acids and alcohols, alkoxylated sorbitan esters, alkylpolyglucosides, PEO-PPO block copolymers, glycerides, and combinations thereof.

21. (canceled)

22. The dispersion composition of claim 18 further comprising an osmoticum.

23. (canceled)

24. (canceled)

25. The dispersion composition of claim 18 wherein the dispersion composition further comprises one or more additional agrochemicals.

26. The dispersion composition of claim 18 wherein the concentration of the polynucleotide is at least about 0.00001 wt. % and/or
wherein the concentration of the surfactant is at least about 0.001 wt. %.

27.-33. (canceled)

34. A method for delivering a polynucleotide into a plant cell, the method comprising applying the dispersion composition, or dilution thereof, of claim 18 onto a plant and/or a part thereof.

35. A process for preparing a particulate composition, the process comprising:
mixing a carbon quantum dot precursor compound and a cationic polymer comprising one or more amine functional groups and having an average molecular weight of from about 3 kDa to about 15 kDa to form a precursor mixture;
carbonizing the carbon quantum dot precursor compound to form functionalized carbon quantum dots; and
complexing one or more polynucleotides for regulating or modulating of a gene expression in a plant cell with the functionalized carbon quantum dots to form the particulate composition, wherein at least a portion of the functionalized carbon quantum dots have a particle size that is no greater than about 15 nm, no greater than about 12 nm, or no greater than about 10 nm.

36. A process for preparing a particulate composition, the process comprising:
carbonizing a carbon quantum dot precursor compound to form carbon quantum dots;
mixing the carbon quantum dots with a cationic polymer comprising one or more amine functional groups and having an average molecular weight of from about 3 kDa to about 15 kDa to form functionalized carbon quantum dots; and
complexing one or more polynucleotides for regulating or modulating of a gene expression in a plant cell with the functionalized carbon quantum dots to form the particulate composition, wherein at least a portion of the functionalized carbon quantum dots have a particle size that is no greater than about 15 nm, no greater than about 12 nm, or no greater than about 10 nm.

37.-47. (canceled)


SEQUENCE LISTING (5 sequences)

SEQ ID NO: 1 - RNA, 42 nt, Artificial Sequence
  Synthetic construct
gauaugggcugaauacaaaucuuuguauucagcccauaucgu

SEQ ID NO: 2 - RNA, 48 nt, Artificial Sequence
  Synthetic construct
augccagauguugcuaugacucuuaagagucauagcaacaucuggcau

SEQ ID NO: 3 - RNA, 44 nt, Artificial Sequence
  Synthetic construct
ggcaucaaggugaacuucaaaauugaaguucaccuugaugccgu

SEQ ID NO: 4 - RNA, 42 nt, Artificial Sequence; identical to SEQ ID NO: 1
  Synthetic construct

SEQ ID NO: 5 - DNA, 44 bp, Artificial Sequence
  Synthetic construct
gaatgtctttgcttccatatttgtatggaagcaaagacattcaa